(19) United States
(12) Patent Application Publication
(10) Pub. No.: US 2005/0192468 A1
(43) Pub. Date: Sep. 1, 2005

(54) Hydrocarbon conversion process improvements

(76) Inventors: Jeffrey H. Sherman, Vero Beach, FL (US); Eric W. McFarland, Santa Baraba, CA (US); Michael J. Weiss, Santa Barbara, CA (US); Ivan Marc Lorkovic, Santa Barbara, CA (US); Leroy E. Laverman, Santa Barabara, CA (US); Shouli Sun, Santa Baraba, CA (US); Dieter J. Schaefer, Goleta, CA (US); Galen D. Stucky, Santa Barbara, CA (US); Peter C. Ford, Santa Barbara, CA (US); Philip Grosso, Auburn, CA (US); Ashley W. Breed, Goleta, CA (US); Michael F. Doherty, Santa Barbara, CA (US)

Correspondence Address: Michael A. O'Neil;Michael A. O'Neil, P.C., Suite 820, 5949 Sherry Lane, Dallas, TX, 75225, US

(21) Appl. No.: 11/098,997

(22) Filed: Apr. 5, 2005

Related U.S. Application Data
(63) Continuation-in-part of application No. 10/430,240, filed on Aug. 19, 2003.
Application No. 10/430,240 is a continuation-in-part of application No. 10/365,346, filed on Feb. 12, 2003.
Application No. 10/365,346 is a continuation of application No. 10/298,440, filed on Nov. 19, 2002, now abandoned.
Application No. 10/298,440 is a continuation-in-part of application No. 10/208,068, filed on Jul. 29, 2002, now abandoned.
Application No. 10/208,068 is a continuation-in-part of application No. 10/054,004, filed on Jan. 24, 2002, now Pat. No. 6,486,368.
Application No. 10/054,004 is a continuation-in-part of application No. 09/951,739, filed on Sep. 11, 2001, now Pat. No. 6,465,696.
Application No. 09/951,739 is a continuation-in-part of application No. 09/886,078, filed on Jun. 20, 2001, now Pat. No. 6,472,572.
(60) Provisional application No. 60/559,844, filed on Apr. 6, 2004.

(51) Int. Cl. C07C 17/42

(52) U.S. Cl. 570/102

(57) ABSTRACT

Improvements in previously disclosed methods of and apparatuses for converting alkanes, alkenes, and aromatics to olefins, alcohols, ethers, and aldehydes includes: safety improvements, use of alternative feedstocks, process simplification, improvements to the halogenation step, improvements to the reproportionation step, improvements to the solid oxide reaction, improvements to solid oxide regeneration, improvements in separations, maintenance, start-up, shut-down, and materials of construction.

### CROSS-REFERENCE TO RELATED APPLICATIONS

This application is a continuation-in-part of application Ser. No. 10/430,240 filed Aug. 19, 2003, currently pending, which is a continuation-in-part of prior application Ser. No. 10/365,346 filed Feb. 12, 2003, currently pending, which is a continuation of prior application Ser. No. 10/298,440 filed Nov. 20, 2002, abandoned, which is a continuation-in-part of prior application Ser. No. 10/208,068, filed Jul. 29, 2002, abandoned, which is a continuation-in-part of prior application Ser. No. 10/054,004 filed Jan. 24, 2002, now U.S. Pat. No. 6,486,368, which is a continuation-in-part of prior application Ser. No. 09/951,739, filed Sep. 11, 2001, now U.S. Pat. No. 6,465,696, which is a continuation-in-part of application Ser. No. 09/886,078 filed Jun. 20, 2001, now U.S. Pat. No. 6,472,572.

### CLAIM OF PRIORITY

Applicant claims priority based on provisional patent application Ser. No. 60/559,844, filed Apr. 6, 2004.

### TECHNICAL FIELD

This invention relates generally methods and apparatuses for synthesizing olefins, alcohols, ethers, and aldehydes from alkanes, alkenes, and aromatics, and more particularly to specific improvements in the methods and apparatuses disclosed in the patents and patent applications identified herein.

### BACKGROUND AND SUMMARY OF THE INVENTION

The present application comprises a continuation-in-part of application Ser. No. 10/430,240, the disclosure of which is incorporated herewith by reference as if set forth herein. The present invention comprises specific improvements in and to the methods and apparatuses disclosed and described in the patents and patent applications identified herein, specifically including:

Safety Improvements;

Use of Alternative Feedstocks;

Process Simplification;

Improvements to the Halogenation Step;

Improvements to the Reproportionation Step;

Improvements to the Solid Oxide Reaction;

Improvements to Solid Oxide Regeneration;

Improvements in Separations;

Maintenance;

Start-up;

Shut-down;

Materials of Construction.

### DETAILED DESCRIPTION

Safety Improvements:

1. The safety of the process may be improved by shipping the halogen in solid halide form. Some specific variations include:
a. The solid may be one of the solids that will be used in the process. One advantage is that separate halide liberation equipment may be avoided. The solid may be regenerated using air, oxygen, and/or oxygen-containing gas in the process equipment.
b. The solid may also be a solid that is not used in the process, but rather a solid which is inexpensive, relatively non-toxic, able to liberate halide at lower temperature, liberates halide with heating rather than oxidation, less hygroscopic, less corrosive in solid form, less corrosive to regenerate, less volatile, more dense, containing a higher halogen content, disposable, and/or otherwise more convenient for shipping. Examples include:
   i. Copper chloride, copper bromide, or copper iodide may be used for the shipment of chlorine, bromine, or iodine due to relatively low cost of copper relative to some other materials.
   ii. Copper halides may be used for shipment for processes in which the reactive solid contains nickel, chromium, lead, cobalt, or other potentially toxic elements.
   iii. Calcium bromide may be used to ship bromine for use in a process using a cobalt-containing metal oxide since cobalt bromide readily forms highly hydrated species.
   iv. Copper chloride, copper bromide, or copper iodide may be used for the shipment of chlorine, bromine, or iodine since copper halides may be easily regenerated with air, oxygen, or oxygen containing gas at temperatures below those required to regenerate other solids.
   v. Copper (II) bromide may be used to ship bromine since bromine may be liberated by heating without oxygen.
   vi. Copper bromide may be used to ship halide for processes using iron-containing materials since iron bromide is hygroscopic, potentially volatile, and potentially corrosive.
   vii. Pure copper halide may be used to ship halide for processes using a supported metal oxide. Such a choice will eliminate the need to transport the inert support.
   viii. Carbon tetrabromide may be used with combustion of this material either in dedicated equipment or within a process unit, generating bromine and carbon dioxide. Such a solid is disposable, thereby eliminating the requirement of returning the solid oxide to a processing facility.
c. The solid may also be a solid that is not used in the process so that the solid used may be shipped in oxide form, which may be more stable, less likely to sinter, dissolve, adsorb (or absorb) water (or other contaminants), or fracture.
d. As the solid used in the process can hold substantially more bromine than the optimum level for use in the process, the solid could be shipped to an operating facility with a relatively high level of bromine that could be used to make up the small amount of bromine that may be lost during normal operation of the process.

2. The safety of the process may be improved by shipping the halogen in liquid halide form. The use of liquid may minimize solids handling operation and associated particulate hazards. Liquid may also be easier to handle.
a. Specifically, boron tribromide may be used with oxidation to boron oxide liberating bromine.

3. The safety of the process may be improved by shipping the halogen in liquid alkyl halide form. The use of liquid halides may minimize solids handling operation and associated particulate hazards. The use of liquid halides may also be easier to handle. The shipment of alkyl halide may be particularly useful for the startup of the plant and may also provide a convenient and safe way to introduce make-up halide.

4. The safety of the process may be improved by the placement of hygroscopic metal halides in selected reactors provided with a sink for water in the event of a process upset. Many metal halides are hygroscopic and will react with water to form hydrates, minimizing corrosion.
a. The metal halides may be selected and placed so that they are molten in the hydrated form and easily removed.
b. The metal halides may be selected based on a low melting temperature so that they can be pumped into the process in the event of an upset.

5. The bromine inventory may be reduced by utilization of the bromine separation apparatus (typically following the regeneration reactor) as the reservoir for bromine for introduction into the alkane bromination reactor or other necessary step involving bromine. This reservoir of liquid bromine will have sufficient capacity to maintain adequate pump priming and allow bromine to be pumped as a liquid rather than using more costly compressors.

6. Reactive metal oxide traps at all process vents for use in normal and emergency operations may be used to insure against release of any and all organic-bromides. These metal oxides may be regenerated to recover bromine.

7. The safety of the process may be enhanced by the use of a solid oxide to dispose of halogenated organic streams and recovery of halide by conversion to carbon dioxide, water, and solid halide. The solid oxide may be regenerated by reacting the solid halide with oxygen, liberating halogen for recycle to the process.
a. One example is the use of CuO or CuZrO₃ to convert vinyl bromide to carbon dioxide, water, coke, and CuBr or CuBrZrO₂.
   Use of Alternate Feedstocks

The above-identified processes may be useful and particularly valuable with feedstocks containing otherwise difficult to separate components. The halogenation chemistry may facilitate the reactive separation of various streams including:

1. The use of steams containing alkane and olefin of the same carbon number.
a. The olefin may be converted with molecular halogen or solid halide to the 1,2-dihalide for use as a feedstock to an epoxide process. The 1,2-dihalide will be easy to separate from the alkane. Examples include:
   i. Converting the propylene in a stream containing propane and propylene to 1,2-dibromopropane and subsequently to propylene oxide. The 1,2-dibromopropane is formed by reacting the mixed hydrocarbon stream with bromine, most preferably at low temperatures where little appreciable reaction with propane occurs. Separation of propane from propylene is required in many existing plants including ethylene plants and is considered one of the most difficult separations in the chemical industry.
b. The olefin may be converted with wet halogen to halohydrin for use as a feedstock to an epoxide process. The halohydrin will be easy to separate from the alkane. Examples include:
   i. Converting the propylene in a stream containing propane and propylene to the bromohydrin and subsequently to propylene oxide. The propylene is converted by passing the hydrocarbon stream through bromine water.
c. The olefin may be converted with hydrogen halide to form the monohalide for use as a feedstock in an olefin, alcohol, epoxide, aldehyde, ketone, or other process. The halide will be easy to separate from the alkane. Examples include:
   i. Converting olefin in a gasoline feed to alkyl halide by reacting with hydrogen halide to form alkyl halide. The alkyl halide can be easily removed, leaving olefin-depleted gasoline.
   ii. Converting butenes to butyl halides in a mixed feed of butanes and butenes. The butyl halides may be coupled to products containing eight carbon atoms for use in gasoline.

2. The use of streams containing branched and linear alkanes resulting in product streams enriched in branched and/or linear molecules.
a. Branched alkanes containing tertiary carbon may be selectively halogenated to alkyl halide and separated, leaving a stream enriched in linear alkane. Examples include:
   i. Depletion of the branched content of detergent-range alkanes by reaction with halide, resulting in greater reactivity with the branched alkanes to branched alkyl halides. Following separation of the branched halides, the remaining stream is enriched in linear alkane. The stream rich in branched halides may be dehydrohalogenated either catalytically or using a solid oxide to create a stream rich in branched olefin for hydroformylation and conversion to branched alcohols.
b. Branched alkanes containing tertiary carbon may be selectively halogenated to alkyl halide. Following separation, the halide stream will inevitably contain some non-branched halides. By selectively dehydrohalogenating the tertiary halides, a stream containing a very high fraction of branched olefins can be separated from the remaining halides. The selective dehydrohalogenation may be conducted thermally, using a catalyst at temperatures below those required for secondary alkyl halide dehydrohalogenation, or using a solid oxide cataloreactant at temperatures below those required for secondary alkyl halide dehydrohalogenation. Dehydrohalogenating the remaining halides will leave a stream enriched in linear olefins.

3. The use of streams containing multiple types of branched molecules resulting in product streams enriched or depleted in molecules containing a certain type or amount of branching:
a. Streams containing linear, mono-branched, and multiply-branched alkanes may be enriched or depleted in multiply-branched product by halogenating the multiply-branched alkanes to multiply-halogenated separation. The multiply-halogenated and/or mono-halide species may be easily separated. Following the desired separation of the non-halogenated, mono-halogenated, and multi-halogenated species, and dehydrohalogenation of the halides, the various streams may be recombined to generate the desired branching composition.
b. Streams containing branched alkanes with and without multiple branching at a single carbon (quaternary carbon) may be depleted in these quaternary carbon-containing species by halogenating the branched alkanes without the quaternary carbon, separating these halides, and dehydrohalogenating. The result will be streams rich in branched olefins without quaternary carbon and alkanes with quaternary carbon.

4. The use of streams containing trace amounts of impurities that are more reactive than the desired alkane reactant:
a. Alkane streams containing aromatics, alcohols, olefins, aldehydes, ketones, sulfides, sulfates, or other reactive molecules may be halogenated at low temperature to selectively halogenate the impurities for removal.
b. Streams of mixed alkanes (e.g. natural gas, refinery streams) may be differentially halogenated based on differing rates of halogenation and subsequently reacted with metal oxides at lower temperatures where the non-halogenated alkanes would pass through without reaction.

5. The use of streams of mixed alkane and olefin in coupling processes. Streams containing alkanes and olefins may be used to produce products of higher carbon number. Several process variations may be employed:
a. A process with:
   i. Olefin hydrohalogenation in the presence of the alkane;
   ii. Separation of the resulting alkyl halide from the alkane;
   iii. Halogenation of Alkane;
   iv. Separation of the resulting alkyl halide from the alkane;
   v. Recycle of alkane;
   vi. Feed of the alkyl halide to the coupling reactor.
b. Several variations of (a) may be employed:
   i. Specifically, step ii (separation of alkyl halide after hydrohalogenation) may or may not be omitted.
   ii. The alkane may or may not be separated from the alkyl halide (step iv).
   iii. Reproportionation chemistry may or may not be employed.
   iv. The olefin and alkane may be separated at the beginning of the process.
   v. Halogenation may precede hydrohalogenation, particularly if high temperature is employed to hinder addition of halogen to the olefin.
   vi. The hydrogen halide used for hydrohalogenation may or may not be the same formed in the halogenation step.
      Process Simplification

1. The halogenation and solid oxide reaction steps may be conducted in the same unit:
a. The halogenation and solid oxide reaction may be simultaneous.
b. The halogenation may occur first by varying the contacting of the hydrocarbon, halogen, and solid oxide.

2. The halogenation, solid oxide reaction, and solid oxide regeneration may be conducted in the same unit by introducing hydrocarbon and oxygen to a solid halide or solid halide-oxide combination. The oxygen will regenerate the solid halide generating hydrocarbon halide and solid oxide, the hydrocarbon halide will react with the oxide, generating product. Variations include:
a. Periodic switching of the direction of feed to the reactor to minimize halogen migration from the reactor.
   Examples include:
   i. Coupling methane to heavier products by cofeeding methane and oxygen over a metal-halide-containing solid.

3. The solid oxide reaction and product separation may be conducted simultaneously when the product is lighter then the reactant the reaction is conducted in a liquid phase reactor under conditions where the product is a vapor and leaves the reactant mixture.

4. Reacting the halide-containing regeneration effluent with olefin to form dihaloalkanes to reduce the energy required for and equipment size in the halide recovery.

5. Reacting the alkane over selected metal-halides in the regeneration step to form the alkyl-halide and a metal-hydride. This would also be a safety improvement and eliminate the need for halogen separation. Materials include but are not limited to halides of boron, nickel, iron, and their mixtures as well as carbon based materials (e.g. C60).

6. Operation of the halogenation process at high halogen:alkane ratio for the feed at temperatures and pressures to maximize the production of monohalo-alkanes at 100% alkane conversion. The alkane feed may be mixed. The products which will contain multiply-halogenenated species and haloacid which may be passed directly over a metal oxide bed to produce a mixture of products dependent upon the reaction conditions will be produced which will be condensed together and separated in the liquid phase by a combination of distillation and phase separation.

7. The use of a hydrogenation step to recover the over-halogenated products by reducing the halogenation to the desired degree. The use of such a step will allow for higher per-pass conversion in the halogenation step. Catalysts may be used, including but not limited to Pd, Pt, Ru, Ni, Au, Cu, and their alloys.

8. Controlling the amount of hydrogen halide added to a metal oxide reactor in order to generate the heat required for an endothermic reaction.

9. The use of hydrogen halide formed in the halogenation step for conversion of byproducts or products into more useful compounds.
a. For example, HBr could be used to hydrobrominate vinylbromide, a common undesirable by-product resulting from HBr elimination from dibromoethane back to same (or any higher vinylbromide equivalent to the corresponding dibromoalkane) . In another example, HBr could be used in the acidic cleavage of ethers into alcohols and alkylbromides, the former increasing the yield of the desired alcohol product and the latter being recycled to the educt stream for reaction on the metal oxide.
   Improvements to the Halogenation Step

1. Improvements in selectivity to desired multiply-halogenated isomers through isomerization of the multiply-halogenated species formed by halogenation. Examples include:
a. Forming dihalides dehydrohalogenating the dihalides, and rehydrohalogenating to form the desired isomers. The rehydrohalogenation may be conducted using process conditions different from the initial halogenation to enhance yield of the desired isomer. The process conditions varied may include temperature, pressure, and catalyst. Some examples include:
   i. Halogenating ethane so that it contains mixed halides including 1,1 and 1,2-dihaloethanes. Dehyrohalogenating the dihaloethanes, and rehydrohalogenating to enrich the 1,2-dihaloethane content.
   ii. Halogenating propane so that it contains mixed halides including 1,1, 2,2, 1,3, and 1,2-dihalopropanes. Dehyrohalogenating the dihalopropanes, and rehydrohalogenating to enrich the 1,2-, 2,2-, 1,3, or 1,1-isomer content.
   iii. Halogenating butane so that it contains mixed dihalides. Dehyrohalogenating, and rehydrohalogenating to enrich the 2,2- or 2,3-isomer content. The 2,2- or 2,3-isomer may be reacted with a metal oxide to make methyl-ethyl ketone.
   iv. Halogenating butane so that it contains mixed tetrahalides. Dehyrohalogenating, and rehydrohalogenating to enrich the 1,2,3,4-isomer content.
   v. Halogenating cyclohexane so that it contains mixed halides including 1,1, 1,2, 1,3, and 1,4-dihaloisomers. Dehyrohalogenating, and rehydrohalogenating to enrich the 1,1, 1,2, 1,3, and 1,4-dihaloisomer content.

2. Enrichment in the primary halide content of a stream of mixed halide isomers by separating primary halides from other halide isomers. Dehydrohalogenating the other isomers, rehydrohalogenating the resulting olefins to produce a stream enriched in primary halide isomers, and returning the resulting stream to the primary halide separation step. Some variations include:
a. Using selective dehydrohalogenation of the non-primary isomers to form easily separated olefin and hydrogen halide.
b. Using distillation to separate the primary and other isomers.
c. Using adsorption to separate the primary and other isomers.
d. Using a shape-selective catalyst to rehydrohalogenate the olefin, enhancing primary halide yield.

3. The use of multiple halogens to create the desired halide isomer. One halogen may be used to halogenate the hydrocarbon and be replaced by another.

4. The use a membrane reactor with halogen on one side and alkane on the other to improve selectivity to the desired halide isomer. This reactor design may improve monohalogenation, dihalogenation, and/or primary halogenation selectivity.

5. Operation of the halogenation reaction at high halogen:alkane ratio to improve conversion may result in unconverted halogen. Photoactivation of the unconverted halogen may be used at low temperature in a solid oxide bed to allow full recovery of all the halogen.

Improvements to the Reproportionation Step

In many processes, the overhalogenated species may be recycled to a point in the process where they are converted to the desired degree of halogenation or less than the desired degree of halogenation. The change in degree of halogenation is termed “reproportionation,” and allows for the use of the carbon and hydrogen in the overhalogenated species, thus reducing feedstock loss and perhaps also allowing greater economic per-pass yield.

Several Improvements Include:
1. A low-temperature reproproportionation step, in which the halogen is redistributed among over-halogenated species, resulting in the formation of optimally halogenated species and additional very highly halogenated species.
   a. An example is a mixture of dibromomethane, tribromomethane, and tetrabromomethane are allowed to react, producing a stream enriched in methyl bromide and tetrabromomethane.
2. A low-temperature reproproportionation step, in which the halogen is redistributed among over-halogenated species, resulting in the formation of optimally halogenated species and additional very highly halogenated species. The yield of optimally halogenated species is maximized by conducting this reproportionation under temperature, pressure, and process conditions such that the reproportionation is conducted in the liquid phase while the optimally halogenated species is predominantly in the vapor phase.
   a. An example is: a mixture of liquid dibromomethane, tribromomethane, and tetrabromomethane are allowed to react in the presence of a catalyst at about 30 C. As the bromine is redistributed and methyl bromide is formed, much of the methyl bromide leaves the solution and enters the vapor phase.
3. The conversion of over-halogenated hydrocarbon to carbon black or other carbon material and halogen. The carbon material may be sold and the halogen may be recycled to the process.
4. The reproportionation of overhalogenated hydrocarbon with another hydrocarbon or halohydrocarbon. Such a process may allow the recovery of the desired hydrocarbon with a loss of a less desirable material.
   a. For example, dibromomethane is reacted with propane to make methyl bromide and brominated propanes. Ideally, one propane molecule can be used to convert eight dibromomethane molecules to methyl bromide. The bromine can be recovered from the brominated propane through thermal decomposition, oxidation, reaction with solid oxide, or other means.
      Improvements to the Solid Oxide Reaction

1. A method of contacting water with alkyl halide and metal oxide in a multi-phase reactor with alkyl halide, solid oxide and optional diluent present at the bottom of the reactor with refluxing water present in a zone above the reactant mixture.

2. The product yield may be increased and process corrosivity may be reduced by conducting the solid oxide reaction in a liquid phase with water present to remove metal halide as it is formed. A specific example is:
a. The reaction is conducted in a vessel containing liquid alkyl halide, liquid water, water vapor, and solid. The water vapor condenses at the top of the reactor or is returned from an external condenser and settles through the metal oxide and alkyl halide containing phase. The water dissolves metal halide as it passes through the alkyl halide phase. The solid oxide may be supported on a plate to keep it out of the liquid water phase. The water and metal halide passes into a separate liquid phase at the bottom of the reactor where some of the water is vaporized. Variations include:
   i. A batch reactor.
   ii. A continuous reactor in which alkyl halide, metal oxide, and water (or steam) are added continuously and metal halide solution is removed continuously to a regeneration reactor where it is dried and regenerated.
   iii. The use of precipitation to remove metal halide from the metal halide solution. By reducing the temperature of the solution, some of the metal halide will precipitate for regeneration. The depleted metal halide solution may be recovered by filtration, centrifugation or other solids-liquid separation methods and recycled to the reactor. Recovered solids can be dried and regenerated to metal oxide and bromine.

3. The liquid phase performance of a reactor may be improved by adding a diluent. The diluent may be, but is not limited to alkanes that are readily separated from the products and reactants.

4. The yield to desired product may be improved by introducing the stream containing hydrocarbon halide to the metal oxide in stages.

5. The yield to desired product may be improved by providing a feed of solid to a fluidized bed reactor that includes some partially or completely spent material. Spent is defined as solid with no remaining oxygen (donation) capacity or bromine capacity.

6. The yield to desired product may be improved by providing a feed of solid to a fluidized bed reactor that includes some partially coked material.

7. The solid oxide reaction may be conducted in a series of switched fixed beds, some of which are undergoing regeneration at any given time.

8. In a process for the production of olefins, the di-halogenated species may be at least partially converted to olefin using certain solids. Some examples include:
a. The reaction of silver metal with 1,2-dibromoethane to form ethylene and silver bromide. The silver bromide may be decomposed to silver and bromine using heat or electromagnetic radiation.
b. The reaction of copper (I) bromide with 1,2-dibromethane to form ethylene and copper(II) bromide. The copper (II) bromide may be decomposed to copper (I) bromide and bromine using heat.
c. The reaction of 1,2-dibromomethane with a metal oxide to form ethylene, carbon dioxide, water, and metal bromide. The metal bromide may be regenerated by reaction with oxygen.
   Improvements to Solid Oxide Regeneration

1. Varying the temperature of solid oxide prior to oxygen introduction to change the particle size of the solid oxide to a more desirable distribution.
a. By raising the temperature, particularly to that above the regeneration onset temperature, prior to introduction of oxygen or air, the metal oxide obtained after regeneration may be reduced in the amount of fines or agglomerates it contains.
b. By introducing oxygen at low temperature, particularly at that below the regeneration onset temperature, the metal oxide obtained after regeneration may be reduced in the amount of fines or agglomerates it contains.

2. Increasing the temperature of solid oxide prior to oxygen introduction to dehydrogenate or desorb adsorbed hydrocarbon, reducing the amount of water and possibly carbon oxides generated in regeneration, thus reducing corrosivity and simplifying halide purification.

3. Performing a separate oxidation, particularly at low temperature, to remove adsorbed hydrocarbon reducing the amount of water and carbon oxides generated in regeneration, thus reducing corrosivity and simplifying halide purification.

4. Introducing water to the solid halide to change the particle size of the resulting solid oxide to a more desirable distribution.
a. The water may be introduced in the gas phase.
b. The water may be introduced in the liquid phase.
c. The water may be introduced concurrently with or prior to the introduction of oxygen.
d. The hydrated solid may be allowed to settle and agglomerate.
e. The hydrated solid may be subjected to intense fluidization to break apart agglomerates.
f. A slurry or aqueous phase may be formed and dried in a manner to form the desired particle size. In particular, spray drying may be used.

5. Dissolving the active metal halide to separate it from impurities, and then converting metal halide to metal oxide.

6. The use of very high temperature regeneration to remove impurities. In particular, chlorine may be removed from metal bromide in this manner.
a. The combination of high temperature with heating of the solid halide prior to oxygen introduction may be particularly useful. In the case of metal bromides, this methodology may allow the removal of chlorine as ClBr or Cl₂.

7. The reduction of the solid halide with hydrogen or other reducing agent to remove impurities. The reduced material may be reoxidized with oxygen, air, or other oxygen containing gas.

Improvements in Separations

1. Separation of halogen from nitrogen, oxygen, and other non-condensibles using solid adsorbents. The solid adsorbents will adsorb the halogen, which can be removed by heating the solid or reducing the pressure. The adsorbents may be, but are not limited to:
a. Molecular sieves;
b. Mesoporous materials;
c. Zeolites;
d. Silica;
e. Alumina;
f. Aluminosilicates;
g. Magnesia;
h. Activated carbon;
i. Metal bromides;
j. Metal oxides;

2. Separation of halogen from nitrogen, oxygen, and other non-condensibles using reactive solid adsorbents. The solid reactive adsorbents will react with the halogen, forming a new chemical composition, from which the halogen can be removed by heating the solid or reducing the pressure, regenerating the solid. The reactive adsorbents may be, but are not limited to:
a. Copper (I) bromide;
b. Iron (II) bromide;
c. Silver bromide;
d. Carbon;
e. Carbon, particularly fullerenes or nano-tubular carbon.

3. Removal of water from halogen by passing the mixed stream over metal halides or metal halide hydrates which may be supported or unsupported. The metal halides will form hydrates and the metal halide hydrates will form more highly hydrated species. The water can be liberated and starting material can be regenerated by heating.

4. Methods of removing trace amounts of halogen from product streams using reactive solids, which may or may not be regenerable. Some specific reactive solids include, but are not limited to:
a. Copper (II) oxide;
b. Silver;
c. Copper;
d. Lithium;
e. Magnesium;
f. Alkali metals.

5. Removal of residual halogen from streams by reaction with olefins. Specific examples include:
a. Reacting with ethylene or propylene to form dihaloalkane, which can be converted to epoxide.
b. Reacting with a heavy multiple-olefin to form highly halogenated species from which the halide can be recovered.

6. Removal of residual hydrocarbon halide from streams by reaction with reactive solids Some specific reactive solids include, but are not limited to:
a. Copper (II) oxide;
b. Silver;
c. Copper;
d. Lithium;
e. Magnesium;
f. Alkali metals.

7. Separation of primary, secondary, and/or tertiary alkyl halides by selective dehydrohalogenation of selected species, separation of the olefin and hydrogen halide from the remaining alkyl halide, and recombination of the hydrogen halide and olefin to form alkyl halides. Examples include, but are not limited to:
a. Separation of primary from secondary alkyl halides by selectively dehydrohalogenating the secondary alkyl halides to olefin and hydrogen halide, separation of the olefin and hydrogen halide from the primary halide, and recombination of the hydrogen halide and olefin to form secondary and possibly primary halide.
b. Separation of primary and secondary alkyl halides from tertiary alkyl halides by selectively dehydrohalogenating the tertiary alkyl halides to olefin and hydrogen halide, separation of the olefin and hydrogen halide from the primary and secondary alkyl halide, and recombination of the hydrogen halide and olefin to form alkyl halide.

8. Removal of sulfur-containing compounds from a hydrocarbon feed by reacting the feed with dry halogen to form sulfur, which can be removed as a solid from the sulfur-depleted hydrocarbon and hydrocarbon halide stream.

9. Removal of sulfur-containing compounds from a hydrocarbon feed by reacting the feed with dry halogen to form sulfur, which can be removed as a solid from the sulfur-depleted hydrocarbon and hydrocarbon halide stream.

10. Removal of carbon dioxide from a stream by reacting with a carbonate-forming material such as calcium oxide. The carbonate may be used in a hydrogen-halide recovery section of the plant. The carbonate will react with hydrogen halide, liberating water and carbon dioxide and producing solid halide, which can be regenerated and recycled to the carbon dioxide separation section.

11. Removal of arsenic, mercury, heavy metal-containing compounds from a hydrocarbon feed by reacting the feed with dry halogen to form solid metal compounds, solid metal halides or halogenated metal hydrocarbons, which can be easily separated.

12. Removing adsorbed product from the solid by rinsing with a compound that is easily separated from the product. Such a rinsing agent may be pentane or other alkane.

13. Removing adsorbed product from the solid by steam distillation.

Maintenance

1. A method of removing coke from reactors by reacting with bromine to form volatile carbon bromides. The carbon bromides may be used in the process in a reproportionation step, thus producing product from the coke.

2. A method of removing coke from reactors by reacting with hydrogen bromide to form volatile hydrocarbon bromides. The carbon bromides may be used in the process in a reproportionation step, thus producing product from the coke.

Start-Up

1. Starting the process with some or all of the solid in the halide or partially halogenated form may provide a number of benefits including:
a. The ability to start the regeneration reactor early in the start up sequence.
b. Reduction in the amount of oxygen carried over into the bromine separation unit.
c. Reduction in the heat generated in the metal oxide reactor or hydrogen halide neutralization step.
d. Reduction in the amount of adsorbed hydrocarbon and thus reduction in the amount of water and carbon dioxide generated in the regeneration unit.
e. Reduction in unfavorable changes in the particle size distribution of the metal oxide.
f. Improvement in the packing of a fixed bed reactor.
g. Providing a hygroscopic metal halide to reactively remove water during upsets.

2. Starting the process with some or all of the solid in the oxide or partially oxygenated form may provide a number of benefits including:
a. The ability to start the solid oxide reactor early in the start up sequence.
b. Reduction in the amount of bromine generated in the regeneration reactor.
c. Reduction in the heat generated in the metal oxide reactor or hydrogen halide neutralization step.
d. Reduction in the amount of adsorbed hydrocarbon and thus reduction in the amount of water and carbon dioxide generated in the regeneration unit.
e. Reduction in unfavorable changes in the particle size distribution of the metal oxide.
f. Improvement in the packing of a fixed bed reactor.
g. Providing a hygroscopic metal halide to reactively remove water during upsets.

3. Starting the process with a solid which has undergone a number of regeneration cycles may offer benefits including:
a. Reduction in byproducts.
b. Improved chemical and thermal stability of reactors.

4. Starting the process with the halogen present in part or completely as alkyl halide may be desirable for a number of reasons including:
a. Less free halogen present during start-up.
b. Ability to start the halide separations section early in the start-up sequence with no hydrogen halide or water present.
   Shut-Down

1. Stopping the process with the halide in metal halide and/or alkyl halide form may improve safety, reduce corrosion, and improve maintenance accessibility.

2. Introducing reactive components into certain sections of the plant may provide a sink for halogen or hydrogen halide, improving safety, reducing corrosion and improving accessibility. An example of such a component is olefin.

Materials of Construction

1. The reactors for alkane halogenation and metathesis consisting of materials to minimize corrosion including but not limited to:
a. Stainless steel;
b. Silicon carbide;
c. Glass lined steel;
d. Titanium;
e. Carbon fiber.

2. Process components operating at temperatures below 300 C. constructed from:
a. Teflon;
b. Glass.

1. For use in conjunction with a process of hydrocarbon conversion of the type including the step of reacting a first reactant with a halogen, a method for improving the safety of the process comprising the steps of:
providing halogen in a solid halide form;
shipping the solid halide from a first location to a second location;
providing a heating source at the second location;
liberating the halogen from the solid halide at the second location using the heating source.

2. For use in conjunction with a process of hydrocarbon conversion of the type including the step of reacting a first reactant with a halogen, a method for improving the safety of the process comprising the steps of:
providing halogen in a liquid halide form;
shipping the liquid halide from a first location to a second location;
providing a heating source at the second location;
liberating the halogen from the liquid halide at the second location using the heating source.

3. For use in conjunction with a process of hydrocarbon conversion of the type including the step of reacting a first reactant with a halogen, a method for improving the safety of the process comprising the steps of:
providing halogen in a liquid alkyl halide form;
shipping the liquid alkyl halide from a first location to a second location;
providing a heating source at the second location;
liberating the halogen from the liquid alkyl halide at the second location using the heating source.

4. For use in conjunction with a process of hydrocarbon conversion of the type including the step of reacting a first reactant with a halogen, a method for improving the safety of the process comprising the steps of:
providing a reactor with a sink;
providing a hygroscopic metal halide;
providing water in the reactor;
placing the hygroscopic metal halide in the reactor for reaction with the water therein;
The reaction of the hygroscopic metal halide with the water resulting in removal of halogen from the hygroscopic metal halide.

5. For use in conjunction with a process of hydrocarbon conversion using bromine as an intermediate to convert alkanes to ethers, a method for improving the safety of the process comprising the steps of:
providing a bromine separation apparatus;
utilizing the bromine separation apparatus to separate bromine into liquid form for use as a reactant in the hydrocarbon conversion process.

6. For use in conjunction with a process of hydrocarbon conversion of the type including the step of reacting a first reactant with a halogen, a method for improving the safety of the process comprising the steps of:
providing a plurality of reactive metal oxide traps;
placing the traps at all process vents; and
utilizing the traps to prevent release of organic bromides during the hydrocarbon conversion process.

7. The method according to claim 6 further comprising the step of regenerating the metal oxide traps to recover bromine therefrom.

8. For use in conjunction with a process of hydrocarbon conversion of the type including the step of reacting a first reactant with a halogen, a method for improving the safety of the process comprising the steps of:
providing a solid oxide;
utilizing the solid oxide to dispose of halogenated organic streams produced in the hydrocarbon conversion process.

9. The method according to claim 8 further comprising the step of utilizing the solid oxide to recover halides by converting bromine used in the hydrocarbon conversion process into carbon dioxide, water, and solid halide.

10. For use in conjunction with a process of hydrocarbon conversion of the type including the step of reacting a first reactant with a halogen, a method for improving the safety of the process comprising the steps of:
providing a hydrocarbon stream containing an alkane and an olefin having the same carbon number;
mixing the hydrocarbon stream with bromide to form a 1,2 dihalide; and
separating the formed 1,2 dihalide from the alkane.

11. For use in conjunction with a process of hydrocarbon conversion requiring a halogenation reaction and a solid halide reaction, a method of improving the hydrocarbon conversion process comprising the steps of:
providing a reactor;
simultaneously carrying out the halogenation reaction and the solid halide reaction in the reactor.

12. For use in conjunction with a process of hydrocarbon conversion requiring a halogenation reaction, a solid halide reaction, and a solid oxide regeneration process, a method of improving the hydrocarbon conversion process comprising the steps of:
providing a reactor;
introducing hydrocarbon and oxygen to a solid halide in the reactor;
utilizing the provided oxygen to regenerate the solid halide thereby regenerating a hydrocarbon and a solid oxide in the reactor;
utilizing the regenerated hydrocarbon to react with the oxide in the reactor to complete the hydrocarbon conversion process.

13. For use in conjunction with a process of hydrocarbon conversion requiring a solid oxide reaction, a method whereby the solid oxide reaction and the hydrocarbon conversion is performed simultaneously comprising the steps of:
providing a reactant which is lighter in weight than the desired hydrocarbon to be produced;
providing a liquid phase reactor;
performing the solid oxide reaction in the liquid phase reactor.

14. For use in conjunction with a process of hydrocarbon conversion of the type including the step of reacting a first reactant with a halogen, a method for reducing the energy required for recovery of halides from an effluent created during the conversion process comprising the step of reacting the effluent with olefin to form dihaloalkanes.

15. For use in conjunction with a process of hydrocarbon conversion of the type including the step of reacting a first reactant with a halogen and requiring regeneration process, a method for improving the safety of the process and eliminating the need for a halogen separation process comprising the steps of:
providing a metal halide;
reacting the first reactant with the metal halide during the regeneration process to form an alky-halide and a metal hydride.

16. For use in conjunction with a process of hydrocarbon conversion requiring a halogenation reaction, a method for maximizing the production of monohalo-alkanes at 100% alkane production comprising the step of operating the halogenation reaction at a high halogen to alkane ratio.

17. For use in conjunction with a process of hydrocarbon conversion of the type including the step of reacting a first reactant with a halogen, a method for improving the halogenation process comprising the steps of:
providing a catalyst;
utilizing the catalyst in a hydrogeneration step to reduce the degree of halogenation in order to recover over-halogenated products produced during the halogenation.

18. For use in conjunction with a process of hydrocarbon conversion of the type including the step of reacting a first reactant with a halogen and requiring a metal oxide reaction, a method for improving the process comprising the steps of:
providing a hydrogen halide for use in the metal oxide reaction;
regulating the hydrogen halide introduced in the metal oxide reaction in order to generate heat for an endothermic reaction.

19. For use in conjunction with a process of hydrocarbon conversion of the type including the step of reacting a first reactant with a halogen, the reaction producing a hydrogen halide, a method for improving the process comprising the step of utilizing the produced hydrogen halide in a subsequent reaction to convert chemical byproducts created during the hydrocarbon conversion process into compounds which may be used in subsequent hydrocarbon conversion processes.

20. For use in conjunction with a process of hydrocarbon conversion of the type including the step of reacting a first reactant with a halogen, the halogenation step producing multiply-halogenated isomers, a method for improving the halogenation step comprising the step of isomerizing the multiply-halogenated isomers.

21. For use in conjunction with a process of hydrocarbon conversion of the type including the step of reacting a first reactant with a halogen, a method for improving the halogenation step comprising the step separating primary halides from other halide isomers.

22. For use in conjunction with a process of hydrocarbon conversion of the type including the step of reacting a first reactant with a halogen, a method for improving the halogenation step comprising the steps of:
providing a plurality of halogens;
utilizing one of the plurality of halogens to halogenate the hydrocarbon;
replacing the halogen used to halogenate the hydrocarbon with another of the plurality of halogens.

23. For use in conjunction with a process of hydrocarbon conversion of the type including the step of reacting a first reactant with a halogen, a method for improving the halogenation step comprising the steps of:
providing a membrane reactor having halogen and alkane on opposing sides thereof;
utilizing the membrane reactor during the halogenation to improve the selectivity of a halide isomer produced therefrom.

24. For use in conjunction with a process of hydrocarbon conversion of the type including the step of reacting a first reactant with a halogen, a method for improving the halogenation step comprising the steps of:
performing the halogenation at a high halogen to alkane ratio;
the performance the halogenation at a high halogen to alkane ratio resulting in an amount of unconverted halogen;
providing a solid oxide bed;
photoactivating the unconverted halogen at a low temperature in the solid oxide bed to recover any remaining halogen.

25. For use in conjunction with a process of hydrocarbon conversion of the type including the step of reacting a first reactant with a halogen which results in an over-halogenated species requiring reproportionation, a method for improving the process comprising the step of reacting halogen with the resulting over-halogenated species at low temperatures thereby resulting in the formation of an optimally halogenated species.

26. The method for improving the hydrocarbon conversion process according to claim 25 wherein the reaction of halogen with the over-halogenated species is performed while over-halogenated species in a liquid phase thereby resulting in the optimally halogenated species being formed in a vapor phase.

27. For use in conjunction with a process of hydrocarbon conversion of the type including the step of reacting a first reactant with a halogen which results in an over-halogenated species requiring reproportionation, a method for improving the process comprising the step of converting the over-halogenated species to a carbon material and halogen.

28. For use in conjunction with a process of hydrocarbon conversion to produce a desired hydrocarbon of the type including the step of reacting a first reactant with a halogen which results in an over-halogenated species requiring reproportionation, a method for improving the process comprising the steps of:
providing a hydrocarbon other than the desired hydrocarbon to be produced by the hydrocarbon conversion;
reacting the provided hydrocarbon with the over-halogenated species to recover any loss of the desired hydrocarbon.

29. For use in conjunction with a process of hydrocarbon conversion of the type including the step of reacting a first reactant with a halogen and requiring a solid oxide reaction, a method for improving the solid oxide reaction step of the process comprising the steps of:
providing a multi-phase reactor;
providing alkyl halide and a solid metal oxide in the bottom of the reactor;
providing water in the reactor above the alkyl halide and solid metal oxide;
reacting the water with the alkyl halide and solid metal oxide in a liquid phase;
removing metal halide formed from the resulting reaction.

30. The method for improving the solid reaction step of the hydrocarbon conversion process according to claim 29 wherein the multi-phase reactor provided is a batch reactor.

31. The method for improving the solid reaction step of the hydrocarbon conversion process according to claim 29 wherein a diluent is provided in conjunction with providing alkyl halide and a solid metal oxide in the bottom of the reactor.

32. For use in conjunction with a process of hydrocarbon conversion to produce a desired hydrocarbon of the type including the step of reacting a first reactant with a halogen and requiring a solid oxide reaction, a method for improving the yield of the desired hydrocarbon from solid oxide reaction step of the process comprising the steps of:
providing a hydrocarbon stream containing hydrocarbon halide; and
introducing the hydrocarbon stream to the oxide reaction in stages.

33. For use in conjunction with a process of hydrocarbon conversion to produce a desired hydrocarbon of the type including the step of reacting a first reactant with a halogen and requiring a solid oxide reaction, a method for improving the yield of the desired hydrocarbon from solid oxide reaction step of the process comprising the steps of:
providing a series of switched fixed beds;
performing the solid reaction step in the series of switched fixed beds.

34. For use in conjunction with a process of hydrocarbon conversion of the type including the step of reacting a first reactant with a halogen and requiring a solid oxide reaction, a method for improving the solid oxide reaction step of the process wherein the di-halogenated species created by the reaction of the first reactant with the halogen is partially converted to the olefin using a solid reactant.

35. For use in conjunction with a process of hydrocarbon conversion of the type including the step of reacting a first reactant with a halogen and requiring regeneration of a solid oxide created during the process, a method for improving the solid oxide regeneration step comprising the steps of:
varying the temperature of the created solid oxide prior to introducing oxygen for reaction therewith to generate a metal oxide;
the introduction of oxygen to the solid oxide at varying temperatures resulting in the generated metal oxide containing fewer particles therein.

36. For use in conjunction with a process of hydrocarbon conversion of the type including the step of reacting a first reactant with a halogen and requiring regeneration of a solid oxide created during the process, a method for improving the solid oxide regeneration step comprising the steps of:
increasing the temperature of the created solid oxide prior to introducing oxygen for reaction therewith to generate a metal oxide; and
the introduction of oxygen to the solid oxide at a higher temperature resulting in desorbing adsorbed hydrocarbons.

37. For use in conjunction with a process of hydrocarbon conversion of the type including the step of reacting a first reactant with a halogen and requiring regeneration of a solid oxide created during the process, a method for improving the solid oxide regeneration step comprising the step of providing a separate oxidation prior to the solid oxide regeneration step.

38. For use in conjunction with a process of hydrocarbon conversion of the type including the step of reacting a first reactant with a halogen and requiring regeneration of a solid oxide created during the process, a method for improving the solid oxide regeneration step comprising the steps of:
providing a halogen in a solid halide form;
introducing water to the solid halide;
the introduction of water to the solid halide resulting in an improved particle distribution within the solid oxide created during the process.

39. For use in conjunction with a process of hydrocarbon conversion of the type including the step of reacting a first reactant with a halogen and requiring regeneration of a solid oxide created during the process, a method for improving the solid oxide regeneration step comprising the step of performing the regeneration step at a very high temperature.

40. For use in conjunction with a process of hydrocarbon conversion of the type including the step of reacting a first reactant with a halogen and requiring regeneration of a solid oxide created during the process, a method for improving the solid oxide regeneration step comprising the steps of:
providing a halogen in a solid halide form;
providing a reducing agent; and
reducing the solid halide with the reducing agent to remove impurities therefrom.

41. For use in conjunction with a process of hydrocarbon conversion of the type including the step for reacting a first reactant with a halogen, a method of separating halogen from nitrogen, oxygen, and other non-condensibles comprising adsorbing the halogen in a structure selected from the group consisting of molecular sieves, mesoporous materials, zeolites, silica, alumina, aluminosilicates, magnesia, activated carbon, metal bromides, and metal oxides.

42. For use in conjunction with a process of hydrocarbon conversion of the type including the steps of reacting a first reactant with a halogen, the step of separating halogen from nitrogen, oxygen, and other non-condensibles by reacting the halogen with a reactive adsorbent selected from the group consisting of copper (I) bromide, iron (II) bromide, silver bromide, carbon, carbon fullerenes and nano-tubular carbon; and further including the step of removing the halogen from the reactive adsorbent.

43. For use in conjunction with a process of hydrocarbon conversion of the type including the steps of reacting a first reactant with a halogen, a method removing water from halogen comprising passing the mixed stream of water and halogen over a material selected from the group consisting of metal halides and metal halide hydrates.

44. For use in conjunction with a process of hydrocarbon conversion of the type including the steps of reacting a first reactant with a halogen, a method of removing trace amounts of halogen from product streams comprising directing the product streams through a reactive solid selected from the group consisting of copper (II) oxide, silver, copper, lithium, magnesium, and alkylide metals.

45. For use in conjunction with a process of hydrocarbon conversion of the type including the steps of reacting a first reactant with a halogen, a method of removing halogen from product streams comprising the step of reacting the product streams with a material selected from the group consisting of ethylene, propylene, and heavy multiple-olefin.

46. For use in conjunction with a process of hydrocarbon conversion of the type including the steps of reacting a first reactant with a halogen, a method of removing residual hydrocarbon halide from product streams by reacting the product streams with a material selected from the group consisting of copper (II) oxide, silver, copper, lithium, magnesium, and alkylide metals.

47. For use in conjunction with a process of hydrocarbon conversion of the type including the steps of reacting a first reactant with a halogen, the step of creating primary, secondary, and tertiary alkyl halides comprising the steps of dehydrohalogenation of predetermined species, separation of olefin and hydrogen halide from the remaining alkyl halide, and recombination of the hydrogen halide and olefin to form alkyl halides.

48. For use in conjunction with a process of hydrocarbon conversion of the type including the steps of reacting a first reactant with a halogen, a method of removing sulfur-containing compounds from a hydrocarbon feed comprising the step of reacting the hydrocarbon feed with dry halogen to form solid sulfur.

49. For use in conjunction with a process of hydrocarbon conversion of the type including the steps of reacting a first reactant with a halogen, a method of removing carbon dioxide from product streams comprising the step of reacting product streams with a carbonate-forming material.

50. For use in conjunction with a process of hydrocarbon conversion of the type including the steps of reacting a first reactant with a halogen, a method of removing materials selected from the group consisting of arsenic, mercury, and heavy metal-containing compounds from a hydrocarbon feed by directing the hydrocarbon feed into engagement with a dry halogen.

51. For use in conjunction with a process of hydrocarbon conversion of the type including the steps of reacting a first reactant with a halogen, a method of removing coke from reactors comprising the step of reacting the coke with bromine.

52. For use in conjunction with a process of hydrocarbon conversion of the type including the steps of reacting a first reactant with a halogen, a method of removing coke from reactors comprising the step of reacting the coke with hydrogen bromide.

53. For use in conjunction with a process of hydrocarbon conversion of the type including the steps of reacting a first reactant with a halogen comprising starting the process with at least part of the solids in either the halide, or partially hydrogenated form.

54. For use in conjunction with a process of hydrocarbon conversion of the type including the steps of reacting a first reactant with a halogen comprising starting the process with at least a portion of the solids in either the halide or partially hydrogenated form.

55. For use in conjunction with a process of hydrocarbon conversion of the type including the steps of reacting a first reactant with a halogen comprising starting the process with at least a portion of the solid or in either the oxide or partially oxygenated form.

56. For use in conjunction with a process of hydrocarbon conversion of the type including the steps of reacting a first reactant with a halogen, the step of stopping the process with the halide and either the metal halide or alkyl halide form.

57. For use in conjunction with a process of hydrocarbon conversion of the type including the steps of reacting a first reactant with a halogen, the method of introducing reactive components into zones comprising a sink for halogen or hydrogen halide.

58. For use in conjunction with a process of hydrocarbon conversion of the type including the steps of reacting a first reactant with a halogen, the step of providing a reactor for halogenation of the first reactant selected from the group consisting of stainless steel, silicon carbide, glass lined steel, titanium, carbon fiber, Teflon®, and glass.
